(19) United States
(12) Patent Application Publication
(10) Pub. No.: US 2024/0342357 A1
(43) Pub. Date: Oct. 17, 2024

(54) ASPIRATION SCOPE UNCLOGGING MECHANISM

(71) Applicant: Boston Scientific Scimed, Inc., Maple Grove, MN (US)

(72) Inventors: Richard C. TAH, Milford, MA (US); Nana ADJEI, Worcester, MA (US); Michael S.H. CHU, Brookline, MA (US)

(73) Assignee: Boston Scientific Scimed, Inc., Maple Grove, MN (US)

(21) Appl. No.: 18/636,508

(22) Filed: Apr. 16, 2024

Related U.S. Application Data
(60) Provisional application No. 63/496,542, filed on Apr. 17, 2023.

(51) Int. Cl. A61M 1/00; A61B 1/307; A61B 18/00

(52) U.S. Cl. CPC A61M 1/774; A61B 1/307; A61B 2018/00511

(57) ABSTRACT

Systems are described, including a system that includes an insertion device with a shaft and a distribution adapter with a housing and a piston. The shaft includes irrigation and aspiration channels providing a fluid circulation through a target area of a body within which the shaft is inserted. The housing includes first and second pairs of diametrically opposed ports. A first port of the first pair is connected to an irrigation port and a second port of the first pair is connected to the irrigation channel. A first port of the second pair is connected to an aspiration port coupled to a negative pressure source and a second port of the second pair is connected to the aspiration channel. The piston is slidably received within a cavity of the housing to be movable between default and reverse configurations.

### CROSS-REFERENCE TO RELATED APPLICATIONS

This application claims the benefit of priority to U.S. Provisional Application No. 63/496,542, filed on Apr. 17, 2023, which is incorporated by reference herein in its entirety.

### TECHNICAL FIELD

The present disclosure relates to a system and a method for treating ureteral or kidney stones. In particular, the present disclosure relates to the system for reversing a flow of a fluid circulation through a target area of a body

### BACKGROUND

Insertion devices for facilitating operations within living bodies, including scope devices, may be used in conjunction with devices such as energy devices and/or retrieval devices to remove foreign bodies or bodily debris such as stones, stone fragments, or tissue from within the human body. For example, ureteroscopes are often used in the treatment of kidney and ureteral stones. Ureteroscopes are typically used in conjunction with devices such as guidewires, retrieval devices, and laser fibers.

As an example, urologists will often use a ureteroscope in combination with a laser fiber to break up and/or pulverize kidney stones so that resulting fragments, particles and/or debris may be removed from the body. According to one recent technique, a ureteroscope may be configured to provide continuous fluid circulation through a target area (e.g., ureter, kidney) to laser a stone and suction resulting fragments, particles and/or debris therefrom. In some cases, however, large fragments may become lodged or stuck within an aspiration channel of the ureteroscope, thereby preventing further extraction of the debris.

### SUMMARY

The present disclosure relates to a system for reversing a flow of a fluid circulation through a target area of a body. The system includes an insertion device with a shaft. The shaft extends longitudinally from a proximal end to a distal end. The shaft includes an irrigation channel extending therethrough and an aspiration channel extending therethrough. The irrigation and aspiration channels are configured to provide the fluid circulation through the target area of the body within which the distal end of the shaft is to be inserted.

The system also includes a distribution adapter. The adapter includes a housing extending longitudinally from an open first end to a closed second end to define a cavity therein. The housing includes a first pair of diametrically opposed ports and a second pair of diametrically opposed ports. A first port of the first pair of ports is connected to an irrigation port configured to be connected to a fluid source and a second port of the first pair of ports is connected to a proximal end of the irrigation channel of the insertion device. A first port of the second pair of ports is connected to an aspiration port configured to be coupled to a negative pressure source and a second port of the second pair of ports is connected to a proximal end of the aspiration channel of the insertion device.

The adapter includes a piston extending longitudinally and including an irrigation hole extending laterally therethrough, an aspiration hole extending laterally therethrough, a reverse irrigation groove extending helically along an exterior surface thereof from a first end to a second end, and a reverse aspiration groove extending helically along the exterior surface from a first end to a second end. The piston slidably is received within the cavity to be movable between a default configuration, in which the irrigation hole of the piston is aligned with the first pair of ports to form a channel therebetween and the aspiration hole is aligned with the second pair of ports to form a channel therebetween, and a reverse configuration, in which the first end of the irrigation groove is aligned with the first port of the first pair of ports and the second end of the irrigation groove is aligned with the second port of the second pair of ports and the first end of the aspiration groove is aligned with the first port of the second pair of ports and the second end of the aspiration groove is aligned with the second port of the first pair of ports.

In an embodiment, the distribution adapter includes a biasing element biasing the piston toward the default configuration.

In an embodiment, the biasing element is a spring received at the closed second end of the cavity of the housing.

In an embodiment, the distribution adapter includes a cap movably mounted over the open first end of the housing so that pressing the cap toward the closed second end of the housing moves the piston from the biased default configuration toward the reverse configuration.

In an embodiment, the first ports of the first pair of ports and the second pair of ports are longitudinally aligned relative to one another and second ports of the first pair of ports and the second pair of ports are longitudinally aligned relative to one another.

In an embodiment, the a first end of the irrigation groove, a first opening of the irrigation hole, a first end of the aspiration groove, and a first opening of the aspiration hole are longitudinally aligned along a first side of the piston and a second end of the reverse aspiration groove, a second opening of the irrigation hole, a second end of the reverse irrigation groove, and a second opening of the aspiration hole are longitudinally aligned along a second side of the piston opposite the first side.

In an embodiment, the system further includes a handle member within which the distribution adapter is housed, the irrigation port and the aspiration port located along a portion of the handle member.

In an embodiment, the handle member includes a tool port configured to a guide a tool therefrom, through a working channel of the insertion device.

In an embodiment, the insertion device is a ureteroscope.

In addition, the present disclosure relates to a system for reversing a flow of a fluid circulation through a target area of a body. The system includes an insertion device with a shaft extending longitudinally from a proximal end to a distal end. The shaft includes an irrigation channel extending therethrough and an aspiration channel extending therethrough. The irrigation and aspiration channels are configured to provide the fluid circulation through the target area of the body within which the distal end of the shaft is to be inserted.

Also, the system includes a distribution adapter which has a housing extending longitudinally from an open first end to a closed second end to define a cavity therein. The housing includes a pair of irrigation ports diametrically opposed to one another, a pair of reverse irrigation ports diametrically opposed to one another, a pair of aspiration ports diametrically opposed to one another, and a pair of reverse aspiration ports diametrically opposed to one another.

The adapter also includes a piston extending longitudinally and having an irrigation hole, a reverse irrigation hole, an aspiration hole and a reverse aspiration hole extending laterally therethrough. The piston is slidably received within the cavity to be movable between a default configuration, in which the irrigation hole of the piston is aligned with the pair of irrigation ports and the aspiration hole of the piston is aligned with the pair of aspiration ports to form corresponding channels therebetween, and a reverse configuration, in which the reverse irrigation hole is aligned with the pair of reverse irrigation ports and the reverse aspiration hole is aligned with the pair of reverse aspiration ports to form corresponding channels therebetween.

In an embodiment, a first port of each of the pair of irrigation ports and the pair of reverse irrigation ports is configured to be connected to a fluid source, and a first port of each of the pair of aspiration ports and the pair of reverse aspiration ports are configured to be connected to a source of negative pressure.

In an embodiment, a second port of each of the pair of irrigation ports and the pair of reverse aspiration ports are connected to a proximal end of the irrigation channel of the insertion device, and a second port of each of the pair of reverse irrigation ports and the pair of aspiration ports is connected to a proximal end of the aspiration channel of the insertion device.

In an embodiment, the distribution adapter includes a biasing element biasing the piston toward the default configuration.

In an embodiment, the biasing element is a spring received at the closed second end of the cavity of the housing.

In an embodiment, the distribution adapter includes a cap movably mounted over the open first end of the housing so that pressing the cap toward the closed second end of the housing moves the piston from the biased default configuration toward the reverse configuration.

In addition, the present disclosure relates to a method for treating ureteral or kidney stones. The method includes inserting a scope device into a patient's body so that a distal end of a shaft thereof is in a target area to be treated, the shaft including an irrigation channel extending therethrough and an aspiration channel extending therethrough, the irrigation and aspiration channels configured to provide a fluid circulation through the target area of a body within which the distal end of the shaft is to be inserted; providing a flow of the fluid circulation through the target area, wherein the flow of the fluid circulation is provided when a distribution adapter is in a default configuration in which a piston of the distribution adapter is received within a housing of the distribution adapter so that an irrigation hole of the piston is aligned with a first pair of diametrically opposed ports of the housing to direct a fluid through the first pair of ports to the irrigation channel of the scope device and an aspiration hole of the piston is aligned with a second pair of diametrically opposed ports of the housing to provide aspiration therethrough to the aspiration channel of the scope device; and reversing the flow of the fluid circulation through the target area by my moving the distribution adapter from the default configuration toward a reverse configuration, in which a first end of an irrigation groove extending about and along an exterior of the piston is aligned with a first port of the first pair of ports and a second end of the irrigation groove is aligned with a second port of the second pair of ports so that irrigation is provided therethrough to direct fluid to the aspiration channel of the scope device, and a first end of an aspiration groove extending about and along the exterior of the piston is aligned with a first port of the second pair of ports and a second end of the aspiration groove is aligned with a second port of the first pair of ports to apply aspiration therethrough to the irrigation channel of the scope device.

In an embodiment, the first port of the first pair of ports is connected to an irrigation port of the scope device and the second port of the first pair of ports is connected to a proximal end of the irrigation channel, and wherein the first port of the second pair of ports is connected to an aspiration port of the scope device and the second port of the second pair of ports is connected to a proximal end of the aspiration channel.

In an embodiment, the aspiration groove of the piston and the irrigation groove of the piston extend helically about the piston without intersecting with one another.

In an embodiment, the piston is biased toward the default configuration via a biasing element received within the housing and reversing the flow of the fluid circulation includes pressing a cap movably mounted over an end of the housing relative to the housing so that the piston is moved relative to the housing from the default configuration toward the reverse configuration.

In an embodiment, the method further includes reverting back from the reverse configuration toward the default configuration by releasing a pressure on the cap.

### BRIEF DESCRIPTION OF THE DRAWINGS

FIG. 1 shows a partial cross-sectional view of a system according to an exemplary embodiment of the present disclosure;

FIG. 2 shows a side view of a distribution adapter according to the system of FIG. 1;

FIG. 3 shows a side view of the distribution adapter of FIG. 2, in an exploded configuration;

FIG. 4 shows a side view of a piston of the distribution adapter of FIG. 2;

FIG. 5 shows a side view of the distribution adapter of FIG. 2, in an assembled configuration;

FIG. 6 shows a cross-sectional view of the distribution adapter housed within a handle member according to the system of FIG. 1, in a default configuration;

FIG. 7 shows an enlarged cross-sectional view of a proximal portion of the handle member housing the distribution adapter according to the system of FIG. 1, in a reverse configuration;

FIG. 8 shows a partial cross-sectional view of a system according to an exemplary embodiment of the present disclosure;

FIG. 9 shows a side view of a distribution adapter according to the system of FIG. 8;

FIG. 10 shows a side view of the distribution adapter of FIG. 9, in an exploded configuration;

FIG. 11 shows a side view of a piston of the distribution adapter of FIG. 9;

FIG. 12 shows another side view of the piston of the distribution adapter of FIG. 9;

FIG. 13 shows yet another side view of the piston of the distribution adapter of FIG. 9;

FIG. 14 shows a partially transparent perspective view of the distribution adapter of FIG. 9, in a default configuration;

FIG. 15 shows a partially transparent perspective view of the distribution adapter of FIG. 9, in a reverse configuration;

FIG. 16 shows an enlarged cross-sectional view of a portion of a handle member including the distribution adapter according to the system of FIG. 8; and

FIG. 17 shows an enlarged cross-sectional view of a distal portion of the handle member including a working adapter according to the system of FIG. 8.

### DETAILED DESCRIPTION

The present disclosure may be further understood with reference to the following description and the appended drawings, wherein like elements are referred to with the same reference numerals. The present disclosure relates to systems and methods for ureteroscopies and, in particular, relates to systems and methods for unclogging a ureteroscope that is adapted to include an irrigation channel, via which fluid may be provided to a target area (e.g., kidney), and an aspiration channel, via which aspiration may be applied, so that continuous fluid circulation is provided to transport fragments, particles, and/or debris resulting from lasering a stone, along with any heat resulting from the lasering, out of the body and away from the target area.

The exemplary embodiments describe a system including a distribution adapter configured to reverse a flow of the fluid circulation so that, when the aspiration channel is clogged via, for example, a large stone fragment or a collection of fragments, fluid is provided through the aspiration channel and suctioned via the irrigation channel so that the fragment(s) may be pushed from the aspiration channel back into the target area for continued lasering. It should be noted that although the exemplary embodiments show and describe a system for reversing the flow of fluid circulation through channels of a ureteroscope, it will be understood by those of skill in the art that the systems and methods of the present disclosure may be used to reverse the flow of fluid circulation through a variety of other insertion devices which may be similarly used in conjunction with other energy devices and/or retrieval devices and in other areas of the body. It should also be noted that the terms “proximal” and “distal,” as used herein, are intended to refer to a direction toward (proximal) and away from (distal) a user of the device (e.g., physician).

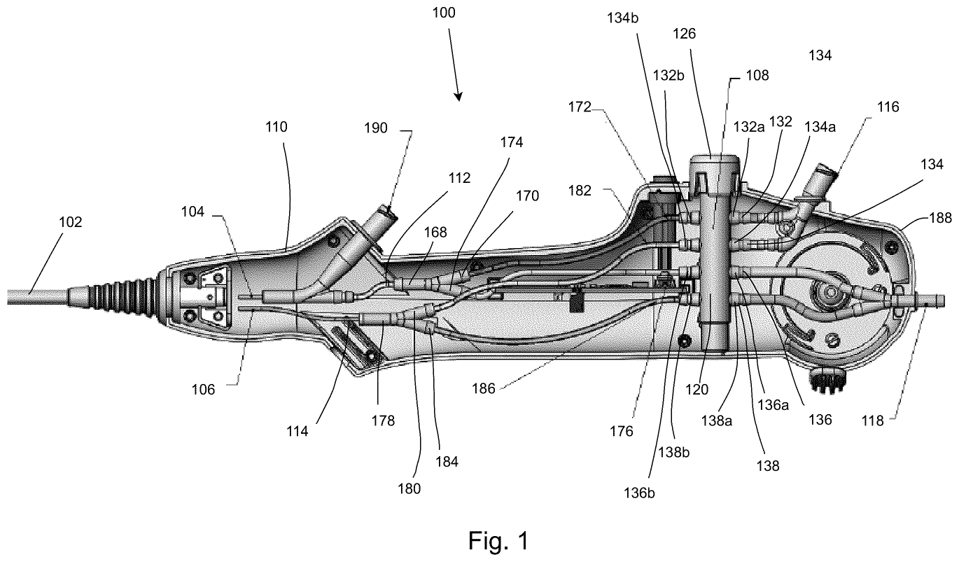

FIGS. 1-7 show a system 100 for reversing a flow of fluid circulation provided via, for example, a scope device 102 adapted to provide a continuous fluid circulation through a target area including a hollow organ or cavity (e.g., ureter, kidney) for suctioning stone fragments, particles, debris, and heat resulting from lasering of a stone in the target area. The scope device 102 may, for example, be adapted to include an irrigation channel 104 via which fluid is provided to the target area and an aspiration channel 106 via which aspiration is provided to suction the stone fragments and heat from the target area. It will be understood by those of skill in the art, however, that the system 100 may be used to reverse a flow of fluid circulation provided by any of a variety insertion devices.

The system 100 comprises a distribution adapter 108 configured to reverse a flow of fluid through the irrigation and aspiration channels 104, 106. In one exemplary embodiment, as shown in FIG. 1, the distribution adapter 108 is housed within a handle member 110 connected to a proximal end of the scope device 102 or other insertion device. A proximal end 112 of the irrigation channel 104 and a proximal end 114 of the aspiration channel 106 extend proximally from the scope device 102 and into the handle member 110 to be connected to the distribution adapter 108, which is movable between a default configuration and a reverse configuration.

In the default configuration, fluid is directed from an irrigation port 116 of the handle member 110 distally through the irrigation channel 104 and aspiration (negative pressure) is directed from an aspiration port 118 of the handle member 110 through the aspiration channel 106 to draw fluid proximally from the distal end of the scope device 102 through the aspiration channel 106 to the aspiration port 118. In the reverse configuration, fluid is supplied from the irrigation port 116 into the aspiration channel 106 so that fluid flows distally through the aspiration channel 106 and aspiration (negative pressure) is applied via the aspiration port 118 to the irrigation channel 104 so that fluid is drawn proximally through the irrigation channel 104 from the distal end of the scope device 102. Thus, in the reverse mode, fluid flows distally through the aspiration channel 106 to push any fragments that may be clogging the aspiration channel 106 out of the scope device 102 and into the target area for further lasering or other treatment.

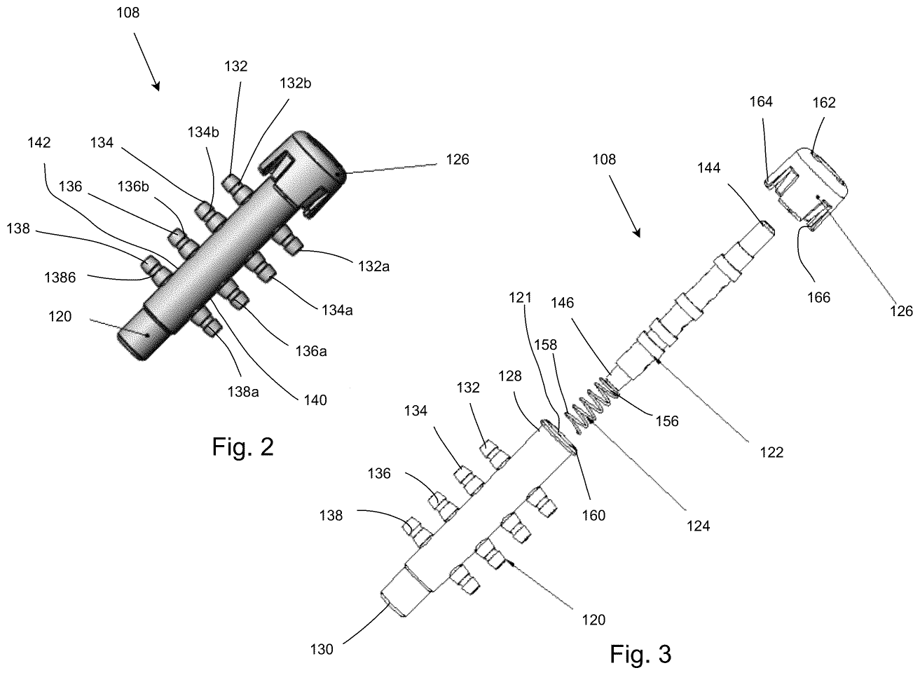

As shown in FIGS. 2-5, the distribution adapter 108 includes a housing 120 within which a piston 122 and a biasing element 124 are received. A cap 126 (e.g., an end cap) is movably coupled to an end of the housing 120 to move the piston 122 relative to the housing 120 between the default configuration and the reverse configuration.

According to an exemplary embodiment, as shown in FIG. 3, the housing 120 extends longitudinally from a first open end 128 to a second closed end 130 to define a cavity 121 therewithin. In an exemplary embodiment, the housing 120 includes a lip 160 protruding radially outwardly from the first end 128 so that the lip 160 is configured to engage the cap 126, as will be described in further detail below. The housing 120 also includes a first pair of diametrically opposed ports 132 configured to facilitate irrigation therethrough, a second pair of diametrically opposed ports 134 configured to facilitate reverse irrigation therethrough, a third pair of diametrically opposed ports 136 configured to facilitate aspiration therethrough, and a fourth pair of diametrically opposed ports 138 configured to facilitate reverse aspiration therethrough.

First ports 132a, 134a, 136a, 138a of each of the first, second, third and fourth pair of ports 132-138 are aligned with one another along a first side 140 of the housing 120 while second ports 132b, 134b, 136b, 138b of each of the first, second, third and fourth pair of ports 132-138 are aligned with one another along a second side 142 of the housing 120 opposite the first side 140. The first port 132a of the first pair of ports 132 and the first port 134a of the second pair of ports 134 are connected to the irrigation port 116. The first port 136a of the third pair of ports 136 and the first port 138a of the fourth pair of ports 138 are connected to the aspiration port 118.

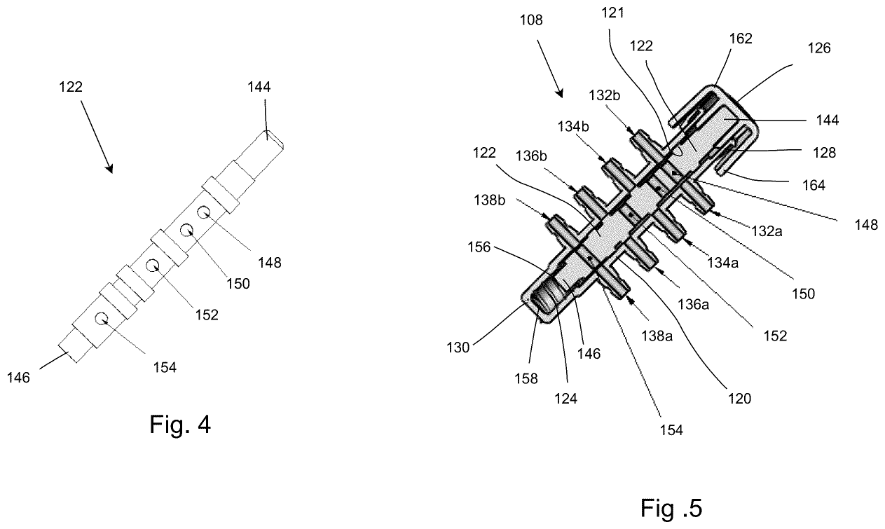

As shown in FIG. 4, the piston 122 is sized and shaped to be slidably received within the cavity 121 of the housing 120. The piston 122 extends within the cavity 121 from a first end 144 to a second end 146 of the piston 122. The piston 122 includes a first irrigation hole 148 extending laterally therethrough, a second reverse irrigation hole 150 extending laterally therethrough, a third reverse aspiration hole 152 extending laterally therethrough, and a fourth aspiration hole 154 extending laterally therethrough. Each of the first, second, third and fourth holes 148-154 of this embodiment extends substantially perpendicular to a longitudinal axis along which the piston 122 extends (i.e., an axis extending between the first end 144 and the second end 146 of the piston 122).

In an exemplary embodiment, the biasing element 124 is received within the housing 120, at the closed second end 130. The biasing element 124 may, for example, be a spring extending from a first end 156 to a second end 158, where the spring is received within the housing 120 with the second end 158 abutting the closed second end 130 of the housing 120 and the second end 146 of the piston 122 abutting the first end 156 of the spring.

The cap 126 is configured to be movably mounted over the first end 128 of the housing 120. In an exemplary embodiment, the cap 126 extends from a closed first end 162 to an open second end 164, the second end 164 including a radially inwardly extending protrusion 166 configured to engage the lip 160 at the first end 128 of the housing 120. When the piston 122 and the biasing element 124 are received within the cavity 121 of the housing 120 and the cap 126 is mounted over the first end 128 of the housing 120 in an assembled configuration, as shown in FIG. 5, the first end 144 of the piston 122 abuts the first end 162 of the cap 126. The cap 126 is movable relative to the housing 120 between a first position, in which the protrusion 166 at the second end 164 engages the lip 160 at the first end 128 of the housing 120, and a second position, in which the cap 126 is moved toward the second end 130 of the housing 120 until a portion of the first end 162 of the cap 126 contacts the first end 128 of the housing 120.

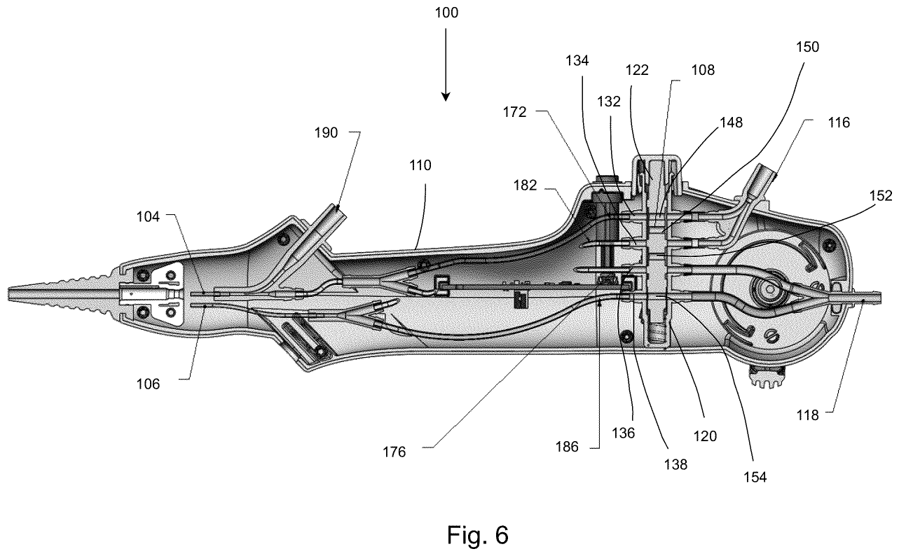

When the cap 126 is in the first position relative to the housing 120 (see FIGS. 5 and 6), the biasing element 124 pushes the piston 122 into the default configuration in which the first hole 148 of the piston 122 is aligned with the first pair of ports 132 to form a channel extending therebetween and the fourth hole 154 of the piston 122 is aligned with the fourth pair of ports 136 to form a channel extending therebetween. In this default configuration, the second and third holes 150, 152 of the piston 122 are not in alignment with any of the ports 132-138 of the housing 120. Thus, in the default configuration, fluid provided via the irrigation port 116 is directed through the first pair of ports 132 and the first hole 148 and aspiration provided via the aspiration port 118 is applied through the fourth pair of ports 138 and the fourth hole 154.

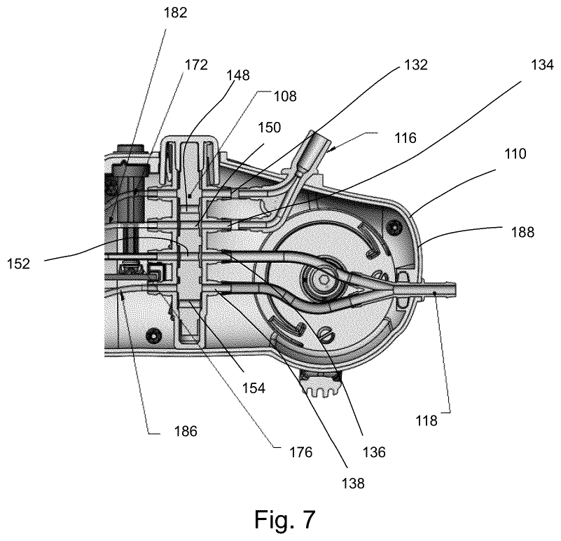

When the cap 126 is moved to the second position relative to the housing 120, the biasing element 124 is compressed and the piston 122 is correspondingly moved to the reverse flow configuration (see FIG. 7) with the second hole 150 of the piston 122 moved into alignment with the second pair of ports 134 of the housing 120 to form a channel therebetween and the third hole 152 of the piston 122 moved into alignment with the third pair of ports 136 to form a channel therebetween. When the piston 122 is in the reverse flow configuration, the first and fourth holes 148, 154 of the piston 122 are moved out of alignment with the first and fourth pair of ports 132, 138, respectively. Thus, in the reverse flow configuration, fluid provided via the irrigation port 116 is directed through the second pair of ports 134 and the second hole 150 while aspiration provided via the aspiration port 118 is applied through the third pair of ports 136 and the third hole.

As described above, in an exemplary embodiment, the proximal ends 112, 114 of the irrigation channel 104 and the aspiration channel 106, respectively, extend proximally from the scope device 102 into the handle member 110. The proximal end 112 of the irrigation channel 104 is connected to, for example, a Y-connector 168, a first branch 170 of which is connected to the second port 132b of the first pair of ports 132 via irrigation tubing 172 and a second branch 174 of which is connected to the second port 136b of the third pair of ports 136 via reverse aspiration tubing 176 so that the irrigation channel 104 may be selectively placed in fluid communication with either the irrigation port 116 via the irrigation tubing 172 or with the aspiration port 118 via the reverse aspiration tubing 176.

The proximal end 114 of the aspiration channel 106 is connected to, for example, a Y-connector 178, a first branch 180 of which is connected the second port 134b of the second pair of ports 134 via reverse irrigation tubing 182 and a second branch 184 of which is connected to the second port 136b of the third pair of ports 136 via the aspiration tubing 186. Thus, the aspiration channel 206 may be selectively placed into fluid communication with either the irrigation port 116 via the reverse irrigation tubing 182 or with the aspiration port 118 via the aspiration tubing 186.

In an exemplary embodiment, the distribution adapter 108 is positioned within the handle member 110 so that the cap 126 extends beyond an exterior of the handle member 110 to be accessible to a user (e.g., physician) of the system 100. While the housing 120 of the distribution adapter 108 may be fixed within the handle member 110, the cap 126 is movable relative to the housing 120 of the distribution adapter 108 and the handle member 110 between the first biased position and the second position. To move the cap 126 from the first position to the second position, the user presses the cap 126 toward the handle member 110, compressing the biasing element 124 and thereby moving the distribution adapter 108 from the default configuration to the reverse flow configuration. In the default configuration, as shown in FIG. 6, fluid supplied to the irrigation port 116 is directed through the first pair of ports 132 via the first hole 148 through the piston 122, through the irrigation tubing 172 into the irrigation channel 104, while aspiration applied to the aspiration port 118 is applied through the fourth pair of ports 138 via the fourth hole 154 through the piston 122 into the aspiration tubing 186 and from there to the aspiration channel 106.

In the reverse configuration, as shown in FIG. 7, fluid supplied to the irrigation port 116 is directed through the second pair of ports 134 via the second hole 150 through the piston 122 into the reverse irrigation tubing 182 and from there into the aspiration channel 106, while aspiration applied to the aspiration port 118 passes through the third pair of ports 138 via the third hole 152 through the piston 122, through the reverse aspiration tubing 176 and from there through the irrigation channel 104. To revert to the default configuration from the reverse configuration, the user simply releases the cap 126 so that the cap 126 and the piston 122 revert back toward the default position relative to the housing 120 under the impetus of the biasing element 124.

According to an exemplary embodiment, the irrigation port 116 is positioned proximate a proximal end 188 of the handle member 110 and extends from the handle member 110 and is configured to be connected to a fluid source. The aspiration port 118 of this embodiment extends from the proximal end 188 of the handle member 110 and is configured to be connected to a source of suction (negative pressure). It will be understood by those of skill in the art, however, that the irrigation port 116 and the aspiration port 118 may be located at any of a variety of locations along the handle member 110 so long as the irrigation port 116 and the aspiration port 118 are connected to the first, second, third and fourth pair of ports 132-138, as described above.

The handle member 110 may include additional features for facilitating use of the scope device 102, as described herein. For example, the handle member 110 may additionally include a tool port 190 via which tools such as, for example, a laser fiber may be inserted into a working channel of the scope device 102 for the lasering of stone in the target area.

According to an exemplary method of use for the system 100, the scope device 102 is inserted into a body lumen via, for example, through a naturally occurring bodily orifice (e.g., urethral opening) or a surgically created opening, to a target area to be treated or observed (e.g., an area in which a target stone or stone fragment to be treated is located). Once the scope device 102 has been inserted to the target area as desired, as would be understood by those skilled in the art continuous fluid circulation may be provided to the target area as the target stone is treated via lasering so that stone fragments, and any heat created via the lasering, may be removed from the body via aspiration.

As described above, the laser device may be inserted to the target area via the tool port 190 to laser the target stone. The continuous fluid circulation in which fluid is supplied to the target area distally through the irrigation channel 104 and withdrawn therefrom proximally via the aspiration channel 106 is provided when the system 100 is in the default configuration. As described above, in the default configuration, the first hole 148 of the piston 122 is aligned with the first pair of ports 132 to form a channel therebetween and the fourth hole 154 of the piston 122 is aligned with the fourth pair of ports 138 to form a channel therebetween. The second and third holes 150, 152 of the piston 122 are not aligned with any of the pair of ports 132-138. Thus, fluid supplied to the system 100 via the irrigation port 116 is delivered to the target area through the first pair of ports 132, the irrigation tubing 172 and the irrigation channel 104, while aspiration provided via the aspiration port 118 draws fluid out of the scope device 102 via the fourth pair of ports 138, the aspiration tubing 186 and the aspiration channel 106.

The user may continue to treat the target stone and any stone fragments, providing continuous fluid circulation to the target area via the default configuration of the system 100, until all of the resulting fragments, particles and/or debris have been removed from the target area. In particular, stone fragments may be further lasered until they are small enough to pass through the aspiration channel 106 and out of the scope device 102. However, if a stone fragment or a collection of fragments becomes lodged or stuck in the aspiration channel 106, the user may move the distribution adapter 108 from the default configuration to the reverse configuration by pressing the cap 126 toward the handle member 110.

In the reverse configuration, the piston 122 is moved relative to the housing 120 so that the first and fourth holes 148, 154 are moved out of alignment with the first pair of ports 132 and the fourth pair of ports 138, respectively, and the second and third holes 150, 152 of the piston 122 are moved into alignment with the second pair of ports 134 and the third pair of ports 136, respectively. Thus, the flow of fluid is reversed so that fluid supplied to the irrigation port 216 is directed through the second pair of ports 134, through the reverse irrigation tubing 182 and distally through the aspiration channel 106 while negative pressure applied to the aspiration port 118 draws fluid out of the body via the third pair of ports 136, the reverse aspiration tubing 176 and the irrigation channel 104.

As would be understood by those skilled in the art, the reverse flow of fluid circulation may be continued until the stone fragment(s) clogging the aspiration channel 106 are pushed distally therefrom, back into the target area for further treatment via, for example, lasering. Once the aspiration channel 106 has been unclogged, the user may release the cap 126 so that the distribution adapter 108 reverts toward the biased default configuration which provides continuous fluid circulation along a default path—distally through the irrigation channel 104 and proximally through the aspiration channel 106.

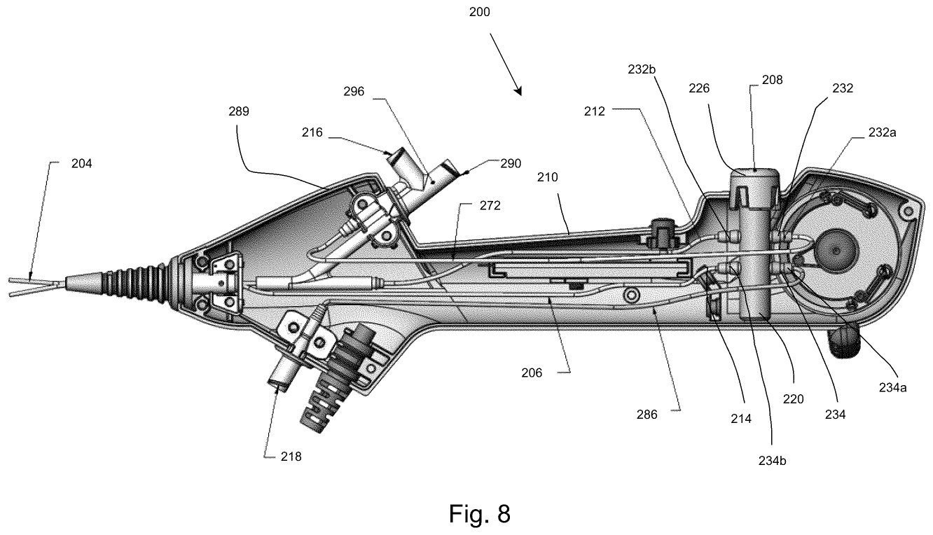

As shown in FIGS. 8-17, a system 200 according to another exemplary embodiment of the present disclosure is, except as indicated below, substantially similar to the system 100 including a distribution adapter 208 configured to reverse a flow of fluid circulation provided via a scope device (not shown) adapted to provide continuous fluid circulation through a target area. As described above with respect to the system 100, and as shown in FIG. 8, the distribution adapter 208 may, in an exemplary embodiment, be housed within a handle member 210 connected to a proximal end of the scope device. A proximal end 212 of an irrigation channel 204 of the scope device and a proximal end 214 of an aspiration channel 206 of the scope device of this embodiment extend proximally from the scope device into the handle member 210 to connect to the distribution adapter 208, which is movable between a default configuration and a reverse configuration in a manner similar to that described above.

In the default configuration, fluid supplied to an irrigation port 216 of the handle member 210 is directed distally through the irrigation channel 204 and aspiration pressure applied to the aspiration port 218 of the handle member 210 is directed through the aspiration channel 206 to draw fluid proximally therethrough. In the reverse configuration, fluid supplied to the irrigation port 216 passes distally through the aspiration channel 206 and aspiration pressure applied to the aspiration port 218 is applied to the irrigation channel 204 to draw fluid proximally therethrough. Thus, the fluid moving distally through the aspiration channel 206 pushes fragments that may be clogging the aspiration channel 206 distally out of the aspiration channel 206 back into the target area for further lasering while this fluid is then aspirated from the target site via the irrigation channel 204.

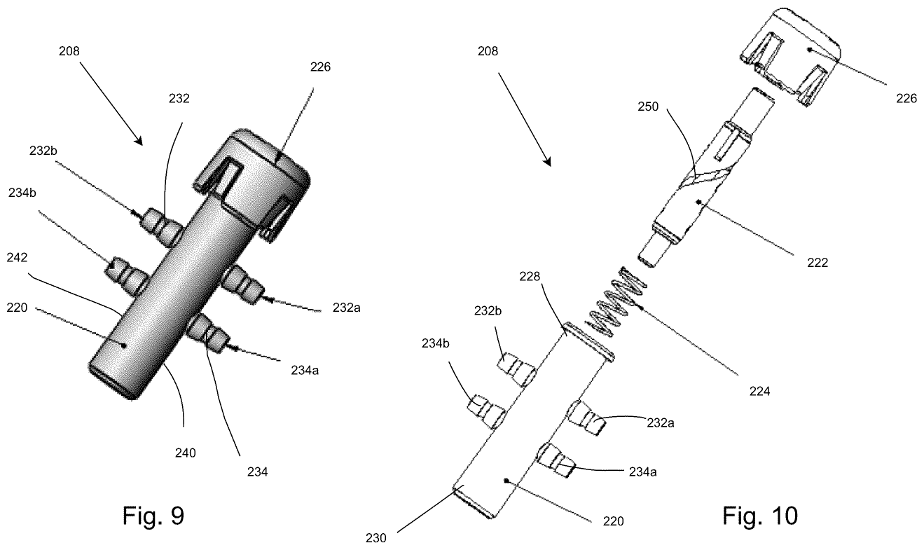

As shown in FIGS. 9-10, similarly to the distribution adapter 108, the distribution adapter 208 includes a housing 220, a piston 222 and a biasing element 224 received within a cavity 221 of the housing 220. A cap 226 is movably mounted over an end of the housing 220 to move the piston 222 relative to the housing 220 between the default configuration and the reverse configuration. The housing 220 is substantially similar to the housing 120 of the distribution adapter 108. The housing 220, however, includes two pairs of ports—a first pair of diametrically opposed ports 232 and a second pair of diametrically opposed ports 234—rather than four pair of ports as described above with respect to the system 100. According to an exemplary embodiment, first ports 232a, 234a of the first pair of ports 232 and the second pair of ports 234, respectively, are longitudinally aligned along a first side of the housing 220 while second ports 232b, 234b of the first pair of ports 232 and the second pair of ports 234, respectively, are longitudinally aligned along a second side 242 of the housing opposite the first side 240. The first port 232a of the first pair of ports 232 is connected to the irrigation port 216 via irrigation tubing 272 and the first port 234a of the second pair of ports 234 is connected to the aspiration port 218 via aspiration tubing 286.

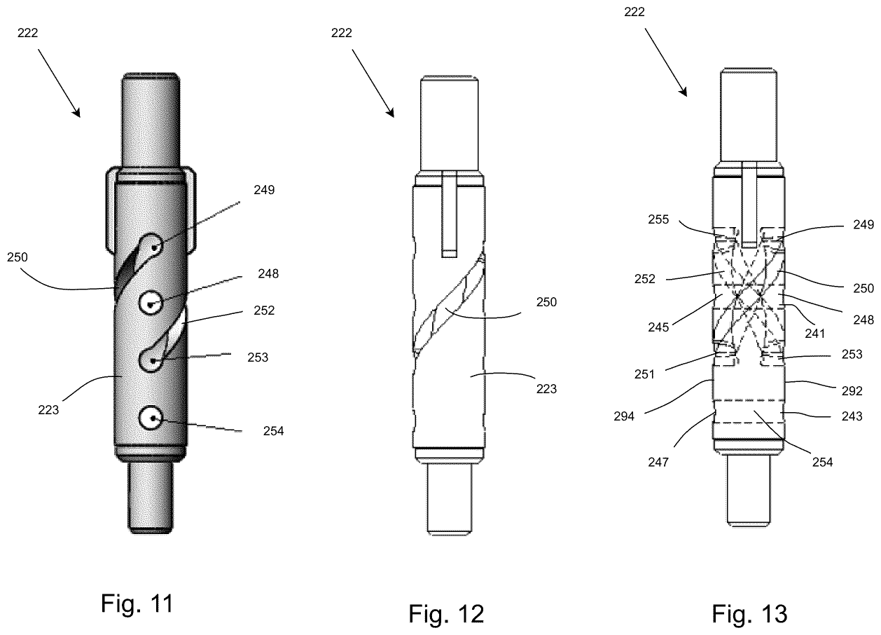

The piston 222, as shown in FIGS. 11-13, of this embodiment is substantially similar to the piston 122, and is sized and shaped to be longitudinally slidably received within the housing 220. The piston 222, however, includes two laterally extending holes-an irrigation hole 248 extending laterally therethrough and an aspiration hole 254 extending laterally therethrough. Similar to the first and fourth holes 148, 154 of the piston 122 of the system 100, the irrigation hole 248 and the aspiration hole 254 are, in this embodiment, substantially perpendicular to a longitudinal axis along which the piston 222 extends.

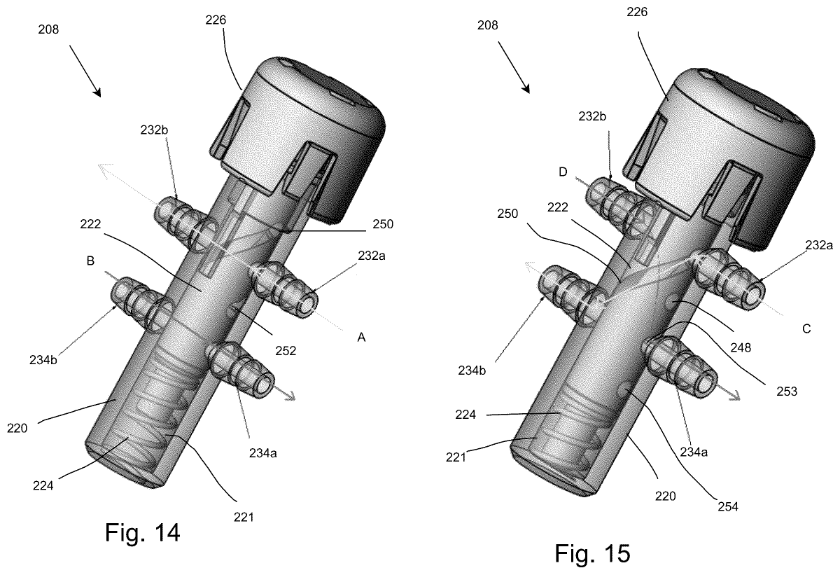

A longitudinal distance between the irrigation hole 248 and the aspiration hole 254 corresponds to a longitudinal distance between the first pair of ports 232 and the second pair of ports 234 so that, when the distribution adapter 208 is in the default configuration, as shown in FIG. 14, the irrigation hole 248 is aligned with and extends between the first pair of ports 232 to form a channel therebetween and the aspiration hole 254 is aligned with and extends between the second pair of ports 234 to form a channel therebetween. As will be described in further detail below, irrigation is provided along a path A, through the channel formed via the first pair of ports 232 and the irrigation hole 248, while aspiration is provided along a path B, through the channel formed via the second pair of ports 234 and the aspiration hole 254.

The piston 222 further includes a reverse irrigation groove 250 and a reverse aspiration groove 252. The reverse irrigation groove 250 extends helically along an exterior 223 of the piston 222 from a first end 249 to a second end 251, the first end 249 extending along a first longitudinal side 292 of the piston 222 and the second end 251 extending along a second longitudinal side 294 of the piston 222 opposite the first side 292. The reverse aspiration groove 252 extends helically along the exterior 223 of the piston 222 from a first end 253 to a second end 255. The first end 253 of the reverse aspiration groove 252 extends along the first side 292 and the second end 255 of the reverse aspiration groove 252 extends along the first side 292. The first end 249 of the reverse irrigation groove 250 is diametrically opposed to the second end 255 of the reverse aspiration groove 252 while the first end 253 of the reverse aspiration groove 252 is diametrically opposed to the second end 251 of the reverse irrigation groove 250. The reverse irrigation groove 250 and the reverse aspiration groove 252 do not intersect along the exterior 223 of the piston 222.

According to an exemplary embodiment, the first end 249 of the irrigation groove 250, a first opening 241 of the irrigation hole 248 along the first side 292, the first end 253 of the reverse aspiration groove 252, and a first opening 243 of the aspiration hole 254 along the first side 292 are all longitudinally aligned along a length of the first side 292 of the piston 222. The second end 255 of the reverse aspiration groove 252, a second end 245 of the irrigation hole 248 along the second side 294, the second end 251 of the reverse irrigation groove 250, and a second hole 247 of the aspiration hole 254 are all longitudinally aligned along a length of the second side 294 of the piston 222. A distance between the first end 249 of the reverse irrigation groove 250 and the first end 253 of the reverse aspiration groove 252 and a distance between the second end 255 of the reverse aspiration groove 252 and the second end 251 of the reverse irrigation groove 250 correspond to the distance between the first and second pair of ports 232, 234.

Thus, when distribution adapter 208 is in the reverse configuration, as shown in FIG. 15, the first end 249 of the reverse irrigation groove 250 is aligned with the first port 232a of the first pair of ports 232 while the second end 251 of the reverse irrigation groove 250 is aligned with the second port 234b of the second pair of ports 234 forming a channel therebetween, and the first end 253 of the reverse aspiration groove 252 is aligned with the first port 234a of the second pair of ports 234 while the second end 255 of the reverse aspiration groove 252 is aligned with the second port 232b of the first pair of ports 232 forming a channel therebetween. Accordingly, in the reverse configuration, fluid supplied to the irrigation port 116 is directed along a path C to the first port 232a of the first pair of ports 232 along the channel formed via the reverse irrigation groove 250 and out of the second port 234b of the second pair of ports 234. Aspiration pressure applied to the aspiration port 218 is directed along a path D, through the first port 234a of the second pair of ports 234, along the channel formed via the reverse aspiration groove 252, and through the second port 232b of the first pair of ports 232.

The biasing element 224 and the cap 226 of this embodiment are substantially similar to the biasing element 124 and the cap 126 of the distribution adapter 108. In particular, the cap 226 is movably mounted over an open first end 228 of the housing 220 while the biasing element 224 is received within the housing 220 at a closed second end 230 to bias the piston 222 toward the default configuration.

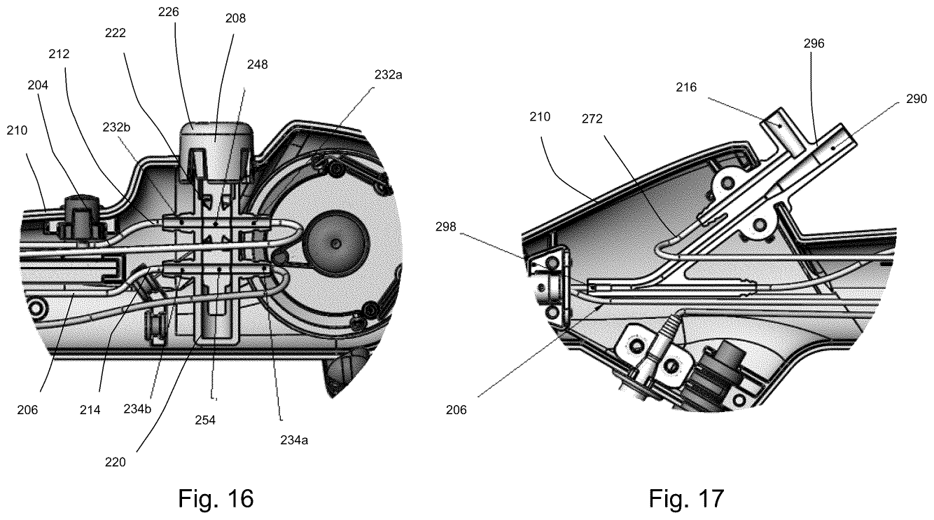

As described above and similarly to the system 100, proximal ends 212, 214 of the irrigation channel 204 and the aspiration channel 206, respectively, extend proximally from the scope device into handle member 210. As shown in FIG. 16, the proximal end 212 of the irrigation channel 204 is connected to the second port 232b of the first pair of ports 232. The proximal end 214 of the aspiration channel 206 is connected to the second port 234b of the second pair of ports 234. Thus, when the distribution adapter 108 is in the default configuration, irrigation fluid is directed from the irrigation port 216, through the irrigation tubing 272, through the first pair of ports 232 via the irrigation hole 248 extending therebetween along the path A, as shown in FIG. 14, and through the irrigation channel 204. Aspiration pressure provided through the aspiration port 218, is directed through the aspiration tubing 286 and through the second pair of ports 234 via the aspiration hole 254 extending therebetween along the path B, as shown in FIG. 14, to apply aspiration pressure through the aspiration channel 206. Accordingly, in the default configuration, continuous fluid circulation is provided to the target area along a path extending distally through irrigation channel 204 and proximally through the aspiration channel 206.

The distribution adapter 208 is moved from the default configuration toward the reverse configuration by pressing the cap 226 toward the second end 230 of the housing 220 to compress the biasing element 224 and correspondingly move the piston 222 longitudinally relative to the housing 220. In the reverse configuration, the irrigation hole 248 and the aspiration hole 254 are moved out of alignment with the first and second pairs of ports 232, 234, respectively, so that the reverse irrigation groove 250 and the reverse aspiration groove 252 are aligned with corresponding ports of the first and second pairs of ports 232, 234, as described above. Thus, in the reverse configuration, fluid circulation through the target area is reversed to be provided along a path opposite the default fluid path-i.e., fluid is supplied distally through the aspiration channel 206 and drawn out of the body proximally through the irrigation channel 204.

In particular, irrigation provided via the irrigation port 216 is applied through the channel formed between the first port 232a of the first pair of ports 232 and the second port 234b of the second pair of ports 234 via the reverse irrigation groove 250 to be provided through the aspiration channel 206 while aspiration pressure provided through the aspiration port 218 is applied through the channel formed between the first port 234a of the second pair of ports 234 and the second port 232b of the first pair of ports 232 via the reverse aspiration groove 252 to provide a negative pressure drawing fluid proximally from the target site in the body through the irrigation channel 204. Similarly to the system 100, when a clog within the aspiration channel 206 has been cleared, as desired, the user may release the cap 226 so that the cap 226 and piston 22 revert to the biased default position relative to the housing 220 and so that fluid flow may revert to the default path (i.e., distally through the irrigation channel 204 and proximally through the aspiration channel 206).

In an exemplary embodiment, and as described above with respect to the system 100, the distribution adapter 208 is housed within the handle member 210 so that the cap 226 is accessible to the user to move the distribution adapter 208 between the default configuration and the reverse configuration, as described above. The handle member 210 may be substantially similar to the handle member 110. In one exemplary embodiment, however, the irrigation port 216 and the aspiration port 218 may be positioned along a distal portion 289 of the handle member 210, similarly to conventional ureteroscopy handles, the irrigation tubing 272 and the aspiration tubing 286 extending along lengths configured to connect the first ports 232a, 234a of the first and second pair of ports 232, 234 to the irrigation port 216 and the aspiration port 218, respectively. It will be understood by those of skill in the art, however, that the irrigation port 216 and the aspiration port 218 may be located along any portion of the handle member 210 so long as the irrigation port 216 and the aspiration port 218 are connected to the distribution adapter 208, as described above.

Similar to the system 100, the handle member 210 may also include a tool port 290 configured to permit passage of tools such as, for example, a laser fiber, to the target area. According to an exemplary embodiment, the irrigation port 216 and the tool port 290 may be connected to one another and formed within a single working adapter 296, as shown in FIG. 17. Each of the irrigation port 216 and the tool port 290 may be connected to and in communication with distinct channels extending through the working adapter 296. For example, tools inserted through the channel of the tool port 290 will be guided through a working channel 298 extending therefrom and into the scope device while the irrigation port 216 includes a channel connected to and in communication with the irrigation tubing 272. It will be understood by those of skill in the art, however, that the irrigation port 216, the aspiration port 218 and the tool port 290 may have any of a variety of configurations so long as each of the ports 216, 218, 290 are configured to receive an appropriate fluid, suction force, or tool, respectively, as described above.

It will be understood by those of skill in the art that the system 200 may be utilized in a manner substantially similarly to the system 100 and may be moved between the default configuration and the reverse configuration to reverse a flow of fluid circulation through a target area, as desired, to unclog an aspiration channel during treatment of a stone within a hollow cavity such as, for example, a ureter or kidney.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather modifications are also covered within the scope of the present invention as defined by the appended claims.

What is claimed is:

1. A system for reversing a flow of a fluid circulation through a target area of a body, comprising:
an insertion device including a shaft extending longitudinally from a proximal end to a distal end, the shaft including an irrigation channel extending therethrough and an aspiration channel extending therethrough, the irrigation and aspiration channels configured to provide the fluid circulation through the target area of the body within which the distal end of the shaft is to be inserted; and
a distribution adapter, including:
a housing extending longitudinally from an open first end to a closed second end to define a cavity therein, the housing including a first pair of diametrically opposed ports and a second pair of diametrically opposed ports, a first port of the first pair of ports connected to an irrigation port configured to be connected to a fluid source and a second port of the first pair of ports connected to a proximal end of the irrigation channel of the insertion device, a first port of the second pair of ports connected to an aspiration port configured to be coupled to a negative pressure source and a second port of the second pair of ports connected to a proximal end of the aspiration channel of the insertion device; and
a piston extending longitudinally and including an irrigation hole extending laterally therethrough, an aspiration hole extending laterally therethrough, a reverse irrigation groove extending helically along an exterior surface thereof from a first end to a second end, and a reverse aspiration groove extending helically along the exterior surface from a first end to a second end, the piston slidably received within the cavity to be movable between a default configuration, in which the irrigation hole of the piston is aligned with the first pair of ports to form a channel therebetween and the aspiration hole is aligned with the second pair of ports to form a channel therebetween, and a reverse configuration, in which the first end of the irrigation groove is aligned with the first port of the first pair of ports and the second end of the irrigation groove is aligned with the second port of the second pair of ports, and the first end of the aspiration groove is aligned with the first port of the second pair of ports and the second end of the aspiration groove is aligned with the second port of the first pair of ports.

2. The system of claim 1, wherein the distribution adapter includes a biasing element biasing the piston toward the default configuration.

3. The system of claim 2, wherein the biasing element is a spring received at the closed second end of the cavity of the housing.

4. The system of claim 2, wherein the distribution adapter includes a cap movably mounted over the open first end of the housing so that pressing the cap toward the closed second end of the housing moves the piston from the biased default configuration toward the reverse configuration.

5. The system of claim 1, wherein first ports of the first pair of ports and the second pair of ports are longitudinally aligned relative to one another, and second ports of the first pair of ports and the second pair of ports are longitudinally aligned relative to one another.

6. The system of claim 1, wherein a first end of the irrigation groove, a first opening of the irrigation hole, a first end of the aspiration groove, and a first opening of the aspiration hole are longitudinally aligned along a first side of the piston and a second end of the reverse aspiration groove, a second opening of the irrigation hole, a second end of the reverse irrigation groove, and a second opening of the aspiration hole are longitudinally aligned along a second side of the piston opposite the first side.

7. The system of claim 1, further comprising a handle member within which the distribution adapter is housed, the irrigation port and the aspiration port located along a portion of the handle member.

8. The system of claim 7, wherein the handle member includes a tool port configured to a guide a tool therefrom, through a working channel of the insertion device.

9. The system of claim 1, wherein the insertion device is a ureteroscope.

10. A system for reversing a flow of a fluid circulation through a target area of a body, comprising:
an insertion device including a shaft extending longitudinally from a proximal end to a distal end, the shaft including an irrigation channel extending therethrough and an aspiration channel extending therethrough, the irrigation and aspiration channels configured to provide the fluid circulation through the target area of the body within which the distal end of the shaft is to be inserted; and
a distribution adapter, including:
a housing extending longitudinally from an open first end to a closed second end to define a cavity therein, the housing including a pair of irrigation ports diametrically opposed to one another, a pair of reverse irrigation ports diametrically opposed to one another, a pair of aspiration ports diametrically opposed to one another, and a pair of reverse aspiration ports diametrically opposed to one another; and
a piston extending longitudinally and including an irrigation hole, a reverse irrigation hole, an aspiration hole and a reverse aspiration hole extending laterally therethrough, the piston slidably received within the cavity to be movable between a default configuration, in which the irrigation hole of the piston is aligned with the pair of irrigation ports and the aspiration hole of the piston is aligned with the pair of aspiration ports to form corresponding channels therebetween, and a reverse configuration, in which the reverse irrigation hole is aligned with the pair of reverse irrigation ports and the reverse aspiration hole is aligned with the pair of reverse aspiration ports to form corresponding channels therebetween.

11. The system of claim 10, wherein a first port of each of the pair of irrigation ports and the pair of reverse irrigation ports is configured to be connected to a fluid source, and a first port of each of the pair of aspiration ports and the pair of reverse aspiration ports are configured to be connected to a source of negative pressure.

12. The system of claim 10, wherein a second port of each of the pair of irrigation ports and the pair of reverse aspiration ports are connected to a proximal end of the irrigation channel of the insertion device, and a second port of each of the pair of reverse irrigation ports and the pair of aspiration ports is connected to a proximal end of the aspiration channel of the insertion device.

13. The system of claim 10, wherein the distribution adapter includes a biasing element biasing the piston toward the default configuration.

14. The system of claim 13, wherein the biasing element is a spring received at the closed second end of the cavity of the housing.

15. The system of claim 13, wherein the distribution adapter includes a cap movably mounted over the open first end of the housing so that pressing the cap toward the closed second end of the housing moves the piston from the biased default configuration toward the reverse configuration.

16. A method for treating ureteral or kidney stones, comprising:
inserting a scope device into a patient's body so that a distal end of a shaft thereof is in a target area to be treated, the shaft including an irrigation channel extending therethrough and an aspiration channel extending therethrough, the irrigation and aspiration channels configured to provide a fluid circulation through the target area of a body within which the distal end of the shaft is to be inserted;
providing a flow of the fluid circulation through the target area, wherein the flow of the fluid circulation is provided when a distribution adapter is in a default configuration in which a piston of the distribution adapter is received within a housing of the distribution adapter so that an irrigation hole of the piston is aligned with a first pair of diametrically opposed ports of the housing to direct a fluid through the first pair of ports to the irrigation channel of the scope device and an aspiration hole of the piston is aligned with a second pair of diametrically opposed ports of the housing to provide aspiration therethrough to the aspiration channel of the scope device; and
reversing the flow of the fluid circulation through the target area by my moving the distribution adapter from the default configuration toward a reverse configuration, in which a first end of an irrigation groove extending about and along an exterior of the piston is aligned with a first port of the first pair of ports and a second end of the irrigation groove is aligned with a second port of the second pair of ports so that irrigation is provided therethrough to direct fluid to the aspiration channel of the scope device, and a first end of an aspiration groove extending about and along the exterior of the piston is aligned with a first port of the second pair of ports and a second end of the aspiration groove is aligned with a second port of the first pair of ports to apply aspiration therethrough to the irrigation channel of the scope device.

17. The method of claim 16, wherein the first port of the first pair of ports is connected to an irrigation port of the scope device and the second port of the first pair of ports is connected to a proximal end of the irrigation channel, and wherein the first port of the second pair of ports is connected to an aspiration port of the scope device and the second port of the second pair of ports is connected to a proximal end of the aspiration channel.

18. The method of claim 16, wherein the aspiration groove of the piston and the irrigation groove of the piston extend helically about the piston without intersecting with one another.

19. The method of claim 16, wherein the piston is biased toward the default configuration via a biasing element received within the housing and reversing the flow of the fluid circulation includes pressing a cap movably mounted over an end of the housing relative to the housing so that the piston is moved relative to the housing from the default configuration toward the reverse configuration.

20. The method of claim 19, further comprising reverting back from the reverse configuration toward the default configuration by releasing a pressure on the cap.
